(19) United States
(12) Patent Application Publication
(10) Pub. No.: US 2012/0035308 A1
(43) Pub. Date: Feb. 9, 2012

(54) METHODS OF MAKING 6-HYDROXYHEXANOPHENONE AND 5-BENZOYLPENTANOIC ACID AND MONO OR DIESTERS THEREOF

(75) Inventors: Kun Wang, Bridgewater, NJ (US); Jihad Mohammed Dakka, Whitehouse Station, NJ (US); Edmund John Mozeleski, Califon, NJ (US); Francisco Manuel Benitez, Houston, TX (US); Lisa Saunders Baugh, Ringoes, NJ (US); Allen David Godwin, Seabrook, TX (US); Diana S. Smirnova, High Bridge, NJ (US); Stephen Zushma, Clinton, NJ (US)

(73) Assignee: EXXONMOBIL RESEARCH AND ENGINEERING COMPANY, Annandale, NJ (US)

(21) Appl. No.: 13/197,253

(22) Filed: Aug. 3, 2011

Related U.S. Application Data
(60) Provisional application No. 61/371,462, filed on Aug. 6, 2010.

(51) Int. Cl. C08K 5/12; C07C 45/28; C08K 5/101; C07C 67/40; C07C 67/08; C07C 69/612; C07C 69/716

(52) U.S. Cl. 524/291; 560/81; 568/320; 560/254; 560/77; 524/290; 524/287

(57) ABSTRACT

Mono- or diester plasticizers of the formula:

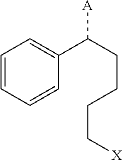

wherein A is either —OC(O)R′ or ═O, and X is either —COC(O)R or —C(O)OR, and R and R′ are C₃ to C₁₃ alkyl, which are the same or different, formed from cyclohexylbenzene and processes of making them.

### CROSS-REFERENCE TO RELATED APPLICATIONS

This is a non-provisional application that claims priority to U.S. Provisional Patent Application No. 61/371,462 filed Aug. 6, 2010, herein incorporated by reference in its entirety.

### FIELD

This disclosure is related to a potential route to non-phthalate, aromatic OXO mono and diester plasticizers.

### BACKGROUND

Plasticizers are incorporated into a resin (usually a plastic or elastomer) to increase the flexibility, workability, or distensibility of the resin. The largest use of plasticizers is in the production of “plasticized” or flexible polyvinyl chloride (PVC) products. Typical uses of plasticized PVC include films, sheets, tubing, coated fabrics, wire and cable insulation and jacketing, toys, flooring materials such as vinyl sheet flooring or vinyl floor tiles, adhesives, sealants, inks, and medical products such as blood bags and tubing, and the like.

Other polymer systems that use small amounts of plasticizers include polyvinyl butyral, acrylic polymers, nylon, polyolefins, polyurethanes, and certain fluoroplastics. Plasticizers can also be used with rubber (although often these materials fall under the definition of extenders for rubber rather than plasticizers). A listing of the major plasticizers and their compatibilities with different polymer systems is provided in “Plasticizers,” A. D. Godwin, in Applied Polymer Science 21st Century, edited by C. D. Craver and C. E. Carraher, Elsevier (2000); pp. 157-175.

Plasticizers can be characterized on the basis of their chemical structure. The most important chemical class of plasticizers is phthalic acid esters, which accounted for 85% worldwide of PVC plasticizer usage in 2002. However, in the recent past there has been an effort to decrease the use of phthalate esters as plasticizers in PVC, particularly in end uses where the product contacts food, such as bottle cap liners and sealants, medical and food films, or for medical examination gloves, blood bags, and IV delivery systems, flexible tubing, or for toys, and the like. For these and most other uses of plasticized polymer systems, however, a successful substitute for phthalate esters has heretofore not materialized.

One such suggested substitute for phthalates are esters based on cyclohexanoic acid. In the late 1990's and early 2000's, various compositions based on cyclohexanoate, cyclohexanedioates, and cyclohexanepolyoate esters were said to be useful for a range of goods from semi-rigid to highly flexible materials. See, for instance, WO 99/32427, WO 2004/046078, WO 2003/029339, WO 2004/046078, U.S. Application No. 2006-0247461, and U.S. Pat. No. 7,297,738.

Other suggested substitutes include esters based on benzoic acid (see, for instance, U.S. Pat. No. 6,740,254, and also co-pending, commonly-assigned, World Patent Publication WO 2009/118261, and polyketones, such as described in U.S. Pat. No. 6,777,514; and also co-pending, commonly-assigned, U.S. patent application Ser. No. 12/058,397, filed Mar. 28, 2008. Epoxidized soybean oil, which has much longer alkyl groups (C₁₆ to C₁₈) has been tried as a plasticizer, but is generally used as a PVC stabilizer. Stabilizers are used in much lower concentrations than plasticizers. Copending and commonly assigned U.S. patent application Ser. No. 12/653,744, filed Dec. 17, 2009, discloses triglycerides with a total carbon number of the triester groups between 20 and 25, produced by esterification of glycerol with a combination of acids derived from the hydroformylation and subsequent oxidation of C₃ to C₉ olefins, having excellent compatibility with a wide variety of resins and that can be made with a high throughput.

U.S. Pat. No. 2,950,320, which is incorporated by reference herein in its entirety, discloses acid catalyzed cleavage of phenylcyclohexane hydroperoxide and more particularly the production of phenol, cyclohexanone and 5-benzoyl pentanol-1 as cleavage products thereof. The phenylcyclohexane hydroperoxide reactant is formed by reacting phenylcyclohexane with NHPI and oxygen. U.S. Pat. No. 2,950,320 fails to disclose or suggest esterification of the resulting 5-benzoyl pentanol-1.

U.S. Provisional Application Ser. No. 61/284,789, filed Dec. 24, 2009, discloses a process for making non-phthalate plasticizers, by acylating an aromatic compound with succinic anhydride to form a keto-acid, and then esterifying the keto-acid with C₄-C₁₃ OXO-alcohols to form a plasticizer compound.

Aoki et al., in an article entitled “One-pot synthesis of phenol and cyclohexanone from cyclohexylbenzene catalyzed by N-hydroxyphthalimide (NHPI)”, Tetrahedron (2005), 61(22), pp. 5219-5222, disclose the N-hydroxyphthalimide (NHPI)-catalyzed one-pot aerobic oxidation of cyclohexylbenzene under oxygen atmosphere at 100° C. for three hours, followed by treatment with 0.3M sulfuric acid at room temperature for two hours to form phenol and cyclohexanone.

To date, none of the prior art compounds or compositions has demonstrated satisfactory equivalence to conventional phthalate plasticizers for use with PVC polymers.

Thus what is needed is a method of making a general purpose non-phthalate plasticizer having suitable melting or chemical and thermal stability, pour point, glass transition, increased compatibility, good performance and low temperature properties.

### SUMMARY

In one aspect, the present application is directed to esters of the formula:

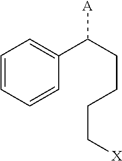

wherein A is either —OC(O)R′ or ═O, and X is either —COC(O)R or —C(O)OR, and R and R′ are C₃ to C₁₃ alkyl, which are the same or different. The esters find use as plasticizers in composition with polymers, such as vinyl chloride resins, polyesters, polyurethanes, ethylene-vinyl acetate copolymer, rubbers, poly(meth)acrylics and combinations thereof.

In another aspect, the esters are diesters of the formula:

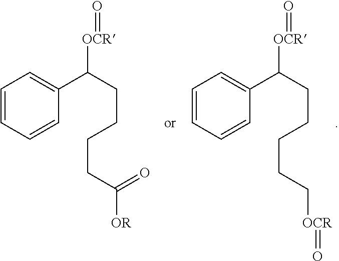

In a preferred embodiment, the diester is one or a mixture of diesters of the formula:

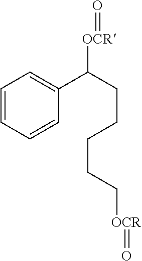

wherein R and R′ are alkyl residues of C₄ to C₁₃ OXO-acids, which are the same or different, and preferably wherein R and R′ are mixed alkyl isomer residues of C₄ to C₉ OXO-acids.

In another preferred embodiment, the diester is one or a mixture of diesters of the formula:

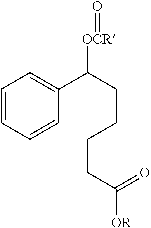

wherein R is alkyl residues of C₄ to C₁₃ OXO-alcohols, and R′ is alkyl residues of C₄ to C₁₃ OXO-acids, and wherein R and R′ can have the same or different carbon numbers, more preferably wherein R and R′ are mixed alkyl isomer residues of the respective OXO-alcohols and OXO-acids, such as wherein R is mixed alkyl isomer residues of C₄ to C₉ OXO-alcohols and R′ is mixed alkyl isomer residues of C₄ to C₉ OXO-acids.

In another embodiment, the esters are monoesters or mixed monoesters of the formula:

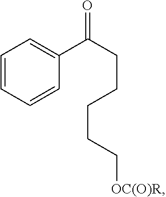

wherein R is C₃ to C₁₃ alkyl which can be the same or different, preferably wherein R is mixed alkyl isomer residues of C₄ to C₁₃ OXO-acids, such as mixed alkyl isomer residues of C₄ to C₉ OXO-acids; or of the formula:

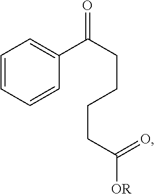

wherein R is C₄ to C₁₃ alkyl which can be the same or different, preferably wherein R is mixed alkyl isomer residues of C₄ to C₁₃ OXO-alcohols, such as mixed alkyl isomer residues of C₄ to C₉ OXO-alcohols.

In another embodiment, the present disclosure is directed to a process for forming 6-hydroxyhexanophenone, comprising (a) oxidizing cyclohexylbenzene in the presence of a molecular oxygen containing gas, such as air or oxygen, and N-hydroxyphthalimide catalyst to form cyclohexylbenzene hydroperoxide; and (b) cleaving the cyclohexyl moiety of said cyclohexylbenzene hydroperoxide in the presence of a polar solvent, such as acetone, nitromethane, nitrobenzene, acetonitrile dimethylsulfoxide, or water, and an acid, such as sulfuric acid, to form 6-hydroxyhexanophenone. Alternatively, 6-hydroxyhexanophenone can be prepared by oxidizing cyclohexylbenzene in the presence of air or other oxygen-containing gases, and N-hydroxyphthalimide (NHPI) together with a metal co-catalyst; wherein the metal can be V, Cr, Mn, Fe, Co, Ni, Cu or mixtures thereof. The metal co-catalyst can be in the form of metal salts such as acetate, acetylacetonate, oxalate, nitrate, sulfate, or chloride.

In a particularly advantageous embodiment, the 6-hydroxyhexanophenone so-formed can be further reacted by (c) oxidizing said 6-hydroxyhexanophenone to form a mono-acid of the formula:

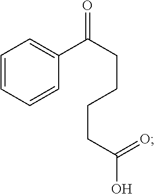

and (d) esterifying said mono-acid with alcohols of the formula ROH, wherein R is C₄ to C₁₃ alkyl which can be the same or different to form monoesters of the formula:

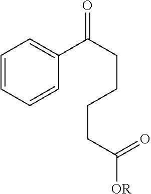

as described above; or by (c) directly esterifying said 6-hydroxyhexanophenone with a first carboxylic acid of the formula RC(O)OH, wherein R is C₄ to C₁₃ alkyl, which can be the same or different, to form monoesters of the formula:

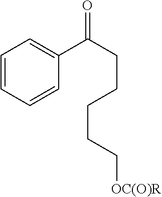

as described above. As stated above these monoesters or mixtures of these monoesters find use as plasticizers in composition with polymers.

Conveniently, the monoesters can be converted to diesters by hydrogenating said monoester to form a compound of the formula:

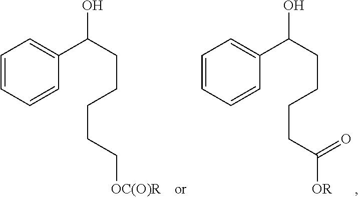

and subsequently esterifying the hydroxyl group with a carboxylic acid of the formula R′C(O)OH, wherein R′ is C₄ to C₁₃ alkyl, which can be the same or different, to form a diester of the formula:

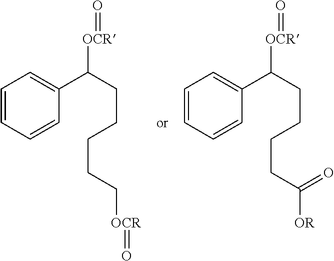

wherein R and R′ are C₃ to C₁₃ alkyl, and can have the same or different carbon numbers. As stated above these diesters or mixtures of these diesters find use as plasticizers in composition with polymers.

### DETAILED DESCRIPTION

All numerical values within the detailed description and the claims herein are modified by “about” or “approximately” the indicated value, and take into account experimental error and variations that would be expected by a person having ordinary skill in the art.

There is an increased interest in developing new plasticizers that are non-phthalates and which possess good plasticizer performance characteristics but are still competitive economically. The present disclosure is directed towards non-phthalate, aromatic ester plasticizers, particularly aromatic OXO-ester plasticizers, that can be made from low cost feeds and employ fewer manufacturing steps in order to meet economic targets. The proposed route to non-phthalate plasticizers of the present disclosure is by esterifying either 6-hydroxyl-hexanopenone or 5-benzoylpentanoic acid, with one or a mixture of C₄ to C₁₃ alcohols and/or C₄ to C₁₃ acids.

In a particularly advantageous embodiment, the aromatic starting material is esterified with OXO-alcohols or OXO-acids, which are mixed linear and branched alcohol/acid isomers, the formation of which is described in more detail below. Esterification can be performed in a simple manner to produce monoesters, or diesters with two identical chains. In the alternative, diesters containing mixed chains are accessible through the use of protected acid reagents or by using alcohol or acid mixtures as reagents.

An “OXO-alcohol” is an organic alcohol, or mixture of organic alcohols, which is prepared by hydroformylating an olefin, followed by hydrogenation to form the alcohols. Typically, the olefin is formed by light olefin oligomerization over heterogenous acid catalysts, which olefins are readily available from refinery processing operations. The reaction results in mixtures of longer-chain, branched olefins, which subsequently form longer chain, branched alcohols, as described in U.S. Pat. No. 6,274,756, incorporated herein by reference in its entirety. The OXO-alcohols consist of multiple isomers of a given chain length due to the various isomeric olefins obtained in the oligomerization process, in tandem with the multiple isomeric possibilities of the hydroformylation step.

An “OXO-acid” is an organic acid, or mixture of organic acids, which is prepared by hydroformylating an olefin, followed by oxidation to form the acids. Typically, the olefin is formed by light olefin oligomerization over heterogenous acid catalysts, which olefins are readily available from refinery processing operations. The reaction results in mixtures of longer-chain, branched olefins, which subsequently form longer-chain, branched acids. The OXO-acids similarly consist of multiple isomers of a given chain length.

An “OXO-ester” is a compound having at least one functional ester moiety within its structure derived from esterification of either an acid or alcohol compound with an OXO-alcohol or OXO-acid, respectively.

Branched aldehydes can be produced by hydroformylation of C₃ to C₁₂ olefins; in turn, some of these olefins have been produced by propylene and/or butene oligomerization over solid phosphoric acid or zeolite catalysts. The resulting C₄ to C₁₃ aldehydes can then be recovered from the crude hydroformylation product stream by fractionation to remove unreacted olefins. These C₄ to C₁₃ aldehydes can then hydrogenated to alcohols (OXO-alcohols) or oxidized to acids (OXO-acids). Single carbon number acids or alcohols can be used in the esterification of the aromatic acids described above, or differing carbon numbers can be used to optimize product cost and performance requirements. The “OXO” technology will provide cost advantaged alcohols and acids. Other options are considered, such as hydroformylation of C₄-olefins to C₅-aldehydes, followed by hydrogenation to C₅-alcohols, or aldehyde dimerization followed by hydrogenation or oxidation to C₁₋₁₀-alcohols or acids.

As discussed above, the resulting C₄ to C₁₃ acids or alcohols can be used individually or together in acid mixtures or alcohol mixtures having different chain lengths, or in isomeric mixtures of the same carbon chain length to make mixed esters to be used as plasticizers. This mixing of carbon numbers and/or levels of branching can be advantageous to achieve the desired compatibility with PVC for the respective core alcohol or acid used for the polar moiety end of the plasticizer, and to meet other plasticizer performance properties. The selected from C₄ to C₁₃ acids or alcohols have an average branching of from 0.2 to 4.0 branches per molecule, more preferably from 0.8 to 3.0 branches per molecule, or from 0.8 to 1.8 branches per molecule. In yet another form, the average branching of the C₃ to C₁₃ branched alkyl groups incorporated into the plasticizers as the residues of the acid or alcohol reagents ranges from 0.2 to 4.0 branches per residue, preferably from 0.8 to 3.0, more preferably from 0.8 to 1.6, more preferably from 1.2 to 1.4 branches per alkyl residue. The starting olefin feed can be C₃═, butenes, C₅═, C₆═, C₇═, C₈═ or C₉═.

Typical branching characteristics of OXO-alcohols and OXO-acids are provided in Tables 1 and 2, below.

TABLE 1
¹³C NMR Branching Characteristics of Typical OXO-Alcohols.

| | | | | | Pendant | Pendant |
|---|---|---|---|---|---|---|
| | | % of α- | β- | Total | Methyls | Ethyls |
| | Avg. | Carbons | Branches | Methyls | per | per |
| OXO- | Carbon | w/ | per | per | Mol- | Mol- |
| Alcohol | No. | Branches^{a} | Molecule^{b} | Molecule^{c} | ecule^{d} | ecule |
| C₄^{e} | 4.0 | 0 | 0.35 | 1.35 | 0.35 | 0 |
| C₅^{f} | 5.0 | 0 | 0.30 | 1.35 | 0.35 | 0 |
| C₆ | — | — | — | — | — | — |
| C₇ | 7.3 | 0 | 0.15 | 1.96 | 0.99 | 0.04 |
| C₈ | 8.6 | 0 | 0.09 | 3.0 | 1.5 | — |
| C₉ | 9.66 | 0 | 0.09 | 3.4 | — | — |
| C₁₀ | 10.2 | 0 | 0.16 | 3.2 | — | — |
| C₁₂ | 12.2 | 0 | — | 4.8 | — | — |
| C₁₃ | 13.1 | 0 | — | 4.4 | — | — |

— Data not available.
^{a}—COH carbon.
^{b}Branches at the —CCH₂OH carbon.
^{c}This value counts all methyl groups, including C₁ branches, chain end methyls, and methyl endgroups on C₂+ branches.
^{d}C₁ branches only.
^{e}Calculated values based on an assumed molar isomeric distribution of 65% n-butanol and 35% isobutanol (2-methylpentanol).
^{f}(Calculated values based on an assumed molar isomeric distribution of 65% n-pentanol, 30% 2-methylbutanol, and 5% 3-methylbutanol.

TABLE 2
¹³C NMR Branching Characteristics of Typical OXO-Acids.

| | Average | | | | % Carbonyls |
|---|---|---|---|---|---|
| OXO- | Carbon | Pendant | Total | Pendant | α to |
| Acid | No. | Methyls^{a} | Methyls^{b} | Ethyls | Branch^{c} |
| C₄^{d} | 4.0 | 0.35 | 1.35 | 0 | 35 |
| C₅^{e} | 5.0 | 0.35 | 1.35 | 0 | 30 |
| C₆ | — | — | — | — | — |
| C₇ | 6.88-7.92 | 0.98-1.27 | 1.94-2.48 | 0.16-0.26 | 11.3-16.4 |
| C₈ | 8.1-8.3 | — | 2.7 | — | 12-15 |
| C₉ | 9.4 | — | n/a | — | 12 |
| C₁₀ | 10.2 | — | n/a | — | 12 |
| C₁₂ | — | — | — | — | — |
| C₁₃ | 12.5 | — | 4.4 | — | 11 |

— Data not available.
^{a}C₁ Branches only.
^{b}Includes methyls on all branch lengths and chain end methyls.
^{c}The “alpha” position in the acid nomenclature used here is equivalent to the alcohol “beta” carbon in Table 1.
^{d}Calculated values based on an assumed molar isomeric distribution of 65% n-butanoic acid and 35% isobutanoic acid (2-methylpentanoic acid).
^{e}Calculated values based on an assumed molar isomeric distribution of 65% n-pentanoic acid, 30% 2-methylbutanoic acid, and 5% 3-methylbutanoic acid..

“Hydroformylating” or “hydroformylation” is the process of reacting a compound having at least one carbon-carbon double bond (an olefin) in an atmosphere of carbon monoxide and hydrogen over a cobalt or rhodium catalyst, which results in addition of at least one aldehyde moiety to the underlying compound. U.S. Pat. No. 6,482,972, which is incorporated herein by reference in its entirety, describes the hydroformylation (OXO) process.

Alternatively, the OXO-acids or OXO-alcohols can be prepared by aldol condensation of shorter-chain aldehydes to form longer chain aldehydes, as described in U.S. Pat. No. 6,274,756, followed by oxidation or hydrogenation to form the OXO-acids or OXO-alcohols, respectively.

“Esterifying” or “esterification” is reaction of a carboxylic acid moiety with an organic alcohol moiety to form an ester linkage. Esterification conditions are well known in the art and include, but are not limited to, temperatures of 0-300° C., and the presence or absence of homogeneous or heterogeneous esterification catalysts such as Lewis or Brønsted acid catalysts.

In general, for every polymer to be plasticized, a plasticizer is required with the correct balance of solvating properties, volatility and viscosity to have acceptable plasticizer compatibility with the resin. In particular, if the 20° C. kinematic viscosity is higher than 150 mm²/sec as measured by the appropriate ASTM test, or alternately if the 20° C. cone-and-plate viscosity is higher than 150 cP, this will affect the plasticizer processability during formulation, and can require heating the plasticizer to ensure good transfer during storage and mixing of the polymer and the plasticizer. Volatility is also a very critical factor which affects the long-term plasticizer formulation stability. Higher volatility plasticizers can migrate from the plastic resin matrix and cause damage to the article. The plasticizer volatility in a resin matrix can be roughly predicted by neat plasticizer weight loss at 220° C. using Thermogravimetric Analysis.

It is known to form cyclohexylbenzene (CHB) by reacting benzene in the presence of hydrogen and a catalyst, and to oxidize CHB to form cyclohexylbenzene hydroperoxide (CHBHP). One potential route to non-phthalate plasticizers is by reacting cyclohexylbenzene hydroperoxide in the presence of an acid and a polar solvent to form an aromatic alcohol, i.e. 6-hydroxylhexanophenone (6-HHP), followed by subsequent esterification with carboxylic acids and/or alcohols.

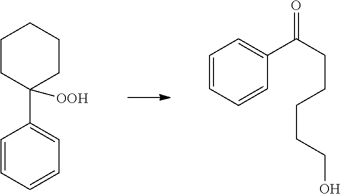

However, prior art processes of forming 6-HHP, such as that disclosed in U.S. Pat. No. 2,950,320, tend to be quite complicated and low in final product yield. Accordingly, the present application is directed to processes of forming 6-HHP both more easily and with greater selectivity than is known in prior art processes.

For example, a process for forming 6-HHP can comprise oxidizing cyclohexylbenzene in the presence of a molecular oxygen containing gas, such as molecular oxygen (O₂) or air, and N-hydroxyphthalimide catalyst to form cyclohexylbenzene hydroperoxide, then cleaving the cyclohexyl moiety of said cyclohexylbenzene hydroperoxide in the presence of a polar solvent and an acid to form 6-hydroxyhexanophenone. Advantageously, the acid is sulfuric acid and the polar solvent is one selected from acetone, nitromethane, nitrobenzene, acetonitrile, dimethylsulfoxide, or water. It is particularly advantageous to use nitromethane as the polar solvent to achieve greatly increased selectivity for 6-HHP, as compared to known prior art processes. Alternatively, 6-hydroxyhexanophenone can be prepared by oxidizing cyclohexylbenzene in the presence of air or other oxygen-containing gases, and N-hydroxyphthalimide (NHPI) together with a metal co-catalyst; wherein the metal can be V, Cr, Mn, Fe, Co, Ni, Cu or mixtures thereof. The metal co-catalyst can be in the form of metal salts such as acetate, acetylacetonate, oxalate, nitrate, sulfate, or chloride.

The resulting 6-HHP can be processed along several different reaction pathways to form non-phthalate plasticizers of the general formula:

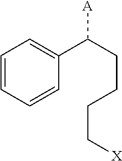

wherein A is either —OC(O)R′ or ═O, and X is either —COC(O)R or —C(O)OR, and R and R′ are C₃ to C₁₃ alkyl, which are the same or different.

In one embodiment, 6-HHP can be reacted with a first carboxylic acid RC(O)OH, preferably a C₄ to C₁₃ OXO-acid, to esterify the pendant hydroxyl group, to form a monoester or mixture of monesters of the formula:

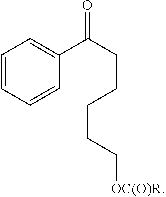

These monoesters can be used as-is as plasticizers for polymers, or further reacted to form diesters by hydrogenation to form a second pendant hydroxyl group, which is subsequently esterified with a second carboxylic acid R′C(O)OH, preferably C₄ to C₁₃ OXO-acid, which can be the same or different from the first carboxylic acid to form an aromatic diester according to the present disclosure. The reaction pathway is set forth below.

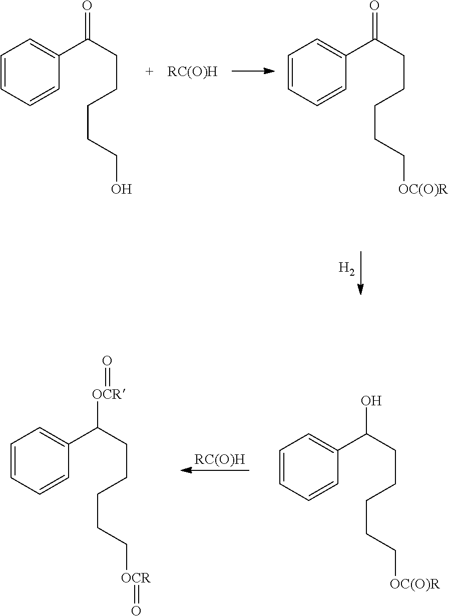

In another embodiment, the 6-HHP is oxidized to the corresponding acid, i.e. 5-benzoylpentanoic acid (5-BPA) using conventional techniques, and the pendant acid group is esterified with an alcohol ROH, preferably C₄ to C₁₃ OXO-alcohol to form a monoester or mixture of monoesters of the formula:

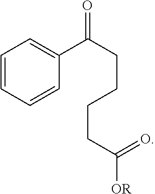

These monoesters can be used as-is as plasticizers for polymers, or further reacted to form diesters by hydrogenation to form a pendant hydroxyl group, which is subsequently esterified with a carboxylic acid R′C(O)OH, preferably C₄ to C₁₃ OXO-acid, which can have the same or different alkyl residue R′ as the alcohol R, to form an aromatic diester according to the present disclosure. The reaction pathway is set forth below.

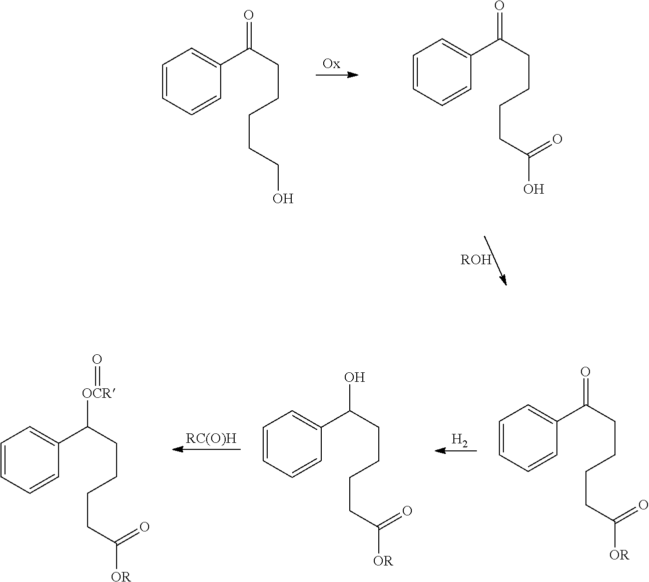

We have found that when C₄ to C₁₃ OXO-alcohols and/or OXO-acids are used as reactants for the esterification reactions described above, the resulting OXO-esters are in the form of relatively high-boiling liquids (having low volatility), which are readily incorporated into polymer formulations as plasticizers.

Advantageously, the present disclosure is directed to an ester or mixture of esters of the formula:

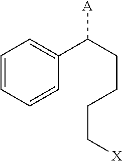

wherein A is either —OC(O)R′ or ═O, and X is either —COC(O)R or —C(O)OR, and R and R′ are C₃ to C₁₃ alkyl, which are the same or different.

Conveniently, the esters are formulated such that R and R′ are alkyl residues of C₄ to C₁₃ OXO-acids or C₄ to C₁₃ OXO-alcohols, preferably C₄ to C₉ OXO-acids or OXO-alcohols, which may be the same or different.

The above described esters can be monoesters of the formula:

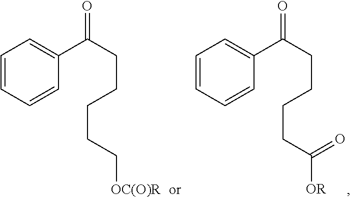

wherein R and R′ are C₃ to C₁₃ alkyl, and can have the same or different carbon numbers.

Likewise, the above described esters can be diesters of the formula:

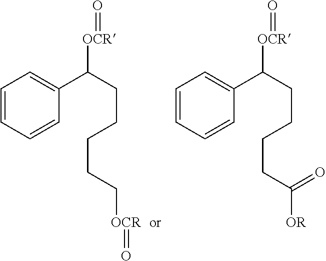

wherein R and R′ are C₃ to C₁₃ alkyl, and can have the same or different carbon numbers.

Any of the esters can have R and R′ which are mixed alkyl isomer residues of C₄ to C₁₃ OXO-acids and/or C₄ to C₁₃ OXO-alcohols, and can be used as plasticizers for polymers, such as vinyl chloride resins, polyesters, polyurethanes, ethylene-vinyl acetate copolymer, rubbers, poly(meth)acrylics and combinations thereof, preferably polyvinylchloride.

In another embodiment, the disclosure is directed to a process for making esters from 6-hydroxyhexanophenone, comprising: (a) oxidizing cyclohexylbenzene in the presence of a molecular oxygen containing gas, such as air or oxygen, and N-hydroxyphthalimide catalyst to form cyclohexylbenzene hydroperoxide; (b) cleaving the cyclohexyl moiety of said cyclohexylbenzene hydroperoxide in the presence of a polar solvent, such as one or more of acetone, nitromethane, nitrobenzene, acetonitrile, dimethylsulfoxide or water and an acid, such as sulfuric acid, to form 6-hydroxyhexanophenone; and either (c1) esterifying said 6-hydroxyhexanophenone with a first carboxylic acid of the formula RC(O)OH, wherein R is C₄ to C₁₃ alkyl, which can be the same or different, to form monoesters of the formula:

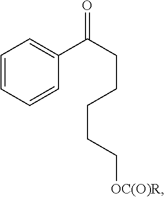

or
(c2) oxidizing said 6-hydroxyhexanophenone to form a mono-acid of the formula:

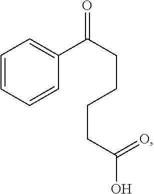

and esterifying said mono-acid with alcohols of the formula ROH, wherein R is C₄ to C₁₃ alkyl which can be the same or different to form monoesters of the formula:

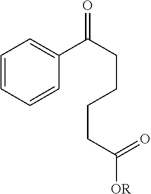

In order to form diesters, the process can optionally further comprise hydrogenating said monoester to form one or more compounds of the formula:

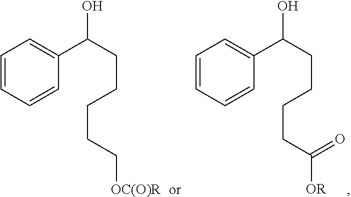

and esterifying the hydroxyl group with a second carboxylic acid of the formula R′C(O)OH, wherein R′ is C₄ to C₁₃ alkyl, which can be the same or different, to form one or more diesters of the formula:

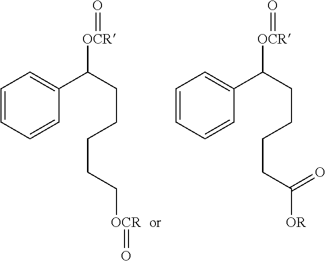

wherein R and R′ are C₃ to C₁₃ alkyl, and can have the same or different carbon numbers.

The following examples are meant to illustrate the present disclosure and inventive processes, and provide where appropriate, a comparison with other methods, including the products produced thereby. Numerous modifications and variations are possible and it is to be understood that within the scope of the appended claims, the disclosure can be practiced otherwise than as specifically described herein.

### EXAMPLES

### Example 1

In a 300-mL autoclave, 150.04 g of cyclohexylbenzene (CHB) and 0.161 g of N-hydroxyphthalimide (NHPI) were loaded. The autoclave was heated to 110° C. under flowing N₂. The N₂ flow was then turned off and air flow turned on (250 cc/min) with vigorous stirring (1000 rpm), and the autoclave was heated at 110° C. for seven hours. The autoclave was then allowed to cool down to room temperature under N₂ and the content collected as the oxidation products. The CHB oxidation product was combined (3007.5 g) and washed with 1% Na₂CO₃ aqueous solution (3×460 mL), followed by a water wash, and the organic phase separated. The pale yellow organic phase (2756.6 g) was dried over 275.7 g anhydrous MgSO₄ to remove residual water. The NHPI level of the final washed product is <10 ppm, compared to 679 ppm in the un-washed sample.

After the NHPI was removed, the CHB oxidation products were divided into 500-mL portions and put into 1-liter plastic bottles. The bottles are placed in a refrigerator held at 5° C. White cyclohexylbenzene hydroperoxide (CHBHP) crystals started to grow in two days. The samples were allowed to sit in the refrigerator for a week. The liquid was decanted, the solid CHBHP crystals were washed with cold pentane and dried under N₂. Yield of CHBHP was 44 g per 500-mL of oxidation products. GC analysis of the CHBHP crystal reveals a purity of 96%. The CHBHP concentration in the mother liquor after the crystallization is 10.5%, compared to 19.3% before crystallization.

An amount of 4.61 g of high purity CHBHP was dissolved in 12.85 g of acetone to make a stock solution. The acetone solution was reacted with 10000 ppm of sulfuric acid in a 5 cc jacketed glass continuous stirred tank reactor (CSTR) fitted with a circulating temperature bath. At the steady state, a residence time of 5 minutes and a temperature of 54° C. were achieved. A 1-cc aliquot was taken, neutralized with 10% Na₂CO₃ solution, and analyzed by GC. [Gas chromatography; analysis was conducted using a Hewlett-Packard 5890 GC equipped with a HP6890 autosampler, a HP flame-ionization detector, and a J&W Scientific DB-1 30 meter column (0.32 micrometer inner diameter, 1 micron film thickness, 100% dimethylpolysiloxane coating). The initial oven temperature was 60° C.; injector temperature 290° C.; detector temperature 300° C.; the temperature ramp rate from 60 to 300° C. was 10° C./minute with a hold at 300° C. for 14 minutes. High yields to phenol and cyclohexanone are achieved (Table 3), but 6-HPP yield was low.]

TABLE 3
Cleavage products from high purity CHBHP using sulfuric
acid in acetone showing low selectivity to 6-HHP

| | Feed | Product |
|---|---|---|
| Component | (wt %, GC) | (wt %, GC) |
| cyclohexanone | 0.05 | 47.25 |
| phenol | 0.04 | 45.31 |
| CHB | 2.24 | 2.21 |
| Ph-1-cyclohexene | 0.15 | 0.50 |
| 4-Ph-cyclohexanol | 0.39 | 0.23 |
| 1-Ph-cyclohexanol | 0.50 | 0.00 |
| Ph-3-cyclohexene | 0.04 | 0.41 |
| CHBHP | 95.83 | 0.00 |
| Peroxide 1 | 0.06 | 0.00 |
| Peroxide 2 | 0.05 | 0.00 |
| Peroxide 3 | 0.00 | 0.00 |
| Peroxide 4 | 0.05 | 0.00 |
| 6-OH-hexanophenone | 0.24 | 3.98 |
| CHBHP conv. (%) | | 100 |
| 6-HHP selectivity (%) | | 4.1 |

### Example 2

The procedure set forth in Example 1 was followed, except that an amount of 2.162 g of high purity CHBHP was dissolved in 3.888 g of nitromethane to make a stock solution. An amount of 4.036 g of the nitromethane solution was added to a 5 cc jacketed glass CSTR reactor fitted with a circulating temperature bath. To the solution 0.2329 g of 5.72 wt % H₂SO₄ solution in nitromethane was added. Instantaneous reaction occurred and the temperature rose to 75° C. A 1 cc aliquot was taken after 1 minute and 3 minutes, neutralized with 10% Na₂CO₃ solution, and analyzed by GC. Yield of 6-HHP increased significantly (Table 4).

TABLE 4
Cleavage products from high purity CHBHP using sulfuric acid
in nitromethane showing increased selectivity to 6-HHP

| | Feed | Product |
|---|---|---|
| Component | (wt %, GC) | (wt %, GC) |
| cyclohexanone | 0.064 | 33.3 |
| phenol | 0.055 | 35.4 |
| CHB | 2.18 | 2.26 |
| Ph-1-cyclohexene | 0.155 | 1.7 |
| 4-Ph-cyclohexanol | 0.353 | 0.18 |
| 1-Ph-cyclohexanol | 0.351 | 0.00 |
| Ph-3-cyclohexene | 0.045 | 0.81 |
| CHBHP | 95.98 | 0.056 |
| Peroxide 1 | 0.06 | 0.00 |
| Peroxide 2 | 0.05 | 0.00 |
| Peroxide 3 | 0.00 | 0.00 |
| Peroxide 4 | 0.05 | 0.036 |
| 6-OH-hexanophenone | 0.194 | 25.3 |
| CHBHP conv. (%) | | 99.9 |
| 6-HHP selectivity (%) | | 26.4 |

It can be seen from the examples above that significant increased yield of 6-HHP can be obtained by the route disclosed here. It is anticipated that by proper choice of solvent, further increases in the selectivity to 6-HHP is possible.

6-HHP can be converted to 5-BPA by a conventional alcohol oxidation technique. Thus a novel process to prepare 6-HHP and 5-BPA from inexpensive starting materials is disclosed, both of which can be used as reagents to make non-phthalate plasticizers.

### Example 3

### Procedure for the Synthesis of the Monoester of 5-Benzoylpentanoic acid+OXO—C₁₀ Alcohols

Into a four-necked 1 liter round bottom flask equipped with an air stirrer, Dean-Stark trap, chilled water cooled condenser, and in and out bubblers for nitrogen were added 5-benzoylpentanoic acid (9142.2 g, 0.69 mole), OXO—C₁₀ alcohols (327.5 g, 2.07 mole) and toluene (149.3 g, 1.6 mole). The reaction mixture was heated at 150° C. for 12 hours and at 150-170° C. for an additional six hours. The total heating time was 18 hours. The reaction mixture was then cooled to room temperature and transferred to a distillation flask. The reaction mixture was fractionated under high vacuum to 0.10 mm. Several distillation cuts contained small amounts of alcohols and acid; these were recombined and the lighter impurities were removed by vacuum distillation. The concentrated product (98.5% purity) was tested as such with no further treatment.

### Example 4

### Neat Properties of OXO—C₁₀ Monoester of 5-Benzoylpentanoic Acid

Thermogravimetric Analysis (TGA) was conducted on the OXO-ester prepared in Example 3 (C₁₀BzC₅) using a TA Instruments AutoTGA 2950HR instrument (25-600° C., 10° C./min, under 60 cc N₂/min flow through furnace and 40 cc N₂/min flow through balance; sample size ˜10 mg). Differential Scanning Calorimetry (DSC) was also performed, using a TA Instruments 2920 calorimeter fitted with a liquid N₂ cooling accessory (−130° C. to 75° C., 10° C./min). Viscosity was measured at 20° C. using an Anton Paar cone-and-plate (25 mm) viscometer (sample size ˜0.1 mL) Table 5 provides a property comparison of the ester versus a common commercial plasticizer (diisononyl phthalate; Jayflex® (DINP), ExxonMobil Chemical Co.). T_{g}s given are midpoints of the second heats.

TABLE 5
Volatility, Viscosity, and Glass Transition Properties of
Plasticizers.

| | TGA | TGA | TGA | TGA Wt | | |
|---|---|---|---|---|---|---|
| | 1% Wt | 5% Wt | 10% Wt | Loss at | | Visc., |
| | Loss | Loss | Loss | 220° C. | DSC T_{g} | 20° C. |
| Material | (° C.) | (° C.) | (° C.) | (%) | (° C.) | (cP) |
| DINP | 184.6 | 215.2 | 228.5 | 6.4 | −79.1 | 99.2 |
| C₁₀BzC₅ | 174.4 | 206.3 | 222.4 | 9.1 | −78.9* | 38.68 |

*DSC also showed a large exotherm at −46.1 and endotherm at −37.1.

### Example 5

### Preparation of PVC Plasticized with OXO—C₁₀ Monoester of 5-Benzoylpentanoic Acid

A 5.85 g portion of the OXO-ester prepared in Example 3 (or comparative commercial plasticizer DINP) was weighed into an Erlenmeyer flask which had previously been rinsed with uninhibited tetrahydrofuran (THF) to remove dust. An 0.82 g portion of a 70:30 by weight solid mixture of powdered Drapex® 6.8 (Crompton Corp.) and Mark® 4716 (Chemtura USA Corp.) stabilizers was added along with a stirbar. The solids were dissolved in 117 mL uninhibited THF. Oxy Vinyls® 240F PVC (11.7 g) was added in powdered form and the contents of the flask were stirred overnight at room temperature until dissolution of the PVC was complete (a PVC solution for preparation of an unplasticized comparative sample was prepared using an identical amount of stabilizer, 100 mL solvent, and 13.5 g PVC). The clear solution was poured evenly into a flat aluminum paint can lid (previously rinsed with inhibitor-free THF to remove dust) of dimensions 7.5″ diameter and 0.5″ depth. The lid was placed into an oven at 60° C. for two hours with a moderate nitrogen purge. The pan was removed from the oven and allowed to cool for a ˜five minute period. The resultant clear film was carefully peeled off of the aluminum, flipped over, and placed back evenly into the pan. The pan was then placed in a vacuum oven at 70° C. overnight to remove residual THF. The dry, flexible film was carefully peeled away and exhibited no oiliness or inhomogeneity. The film was cut into small pieces to be used for preparation of test bars by compression molding (size of pieces was similar to the hole dimensions of the mold plate). The film pieces were stacked into the holes of a multi-hole steel mold plate, pre-heated to 170° C., having hole dimensions 20 mm×12.8 mm×1.8 mm (ASTM D1693-95 dimensions). The mold plate was pressed in a PHI company QL-433-6-M2 model hydraulic press equipped with separate heating and cooling platforms. The upper and lower press plates were covered in Teflon®-coated aluminum foil and the following multistage press procedure was used at 170° C. with no release between stages: (1) three minutes with 1-2 ton overpressure; (2) one minute at 10 tons; (3) one minute at 15 tons; (4) three minutes at 30 tons; (5) release and three minutes in the cooling stage of the press (7° C.) at 30 tons. A knockout tool was then used to remove the sample bars with minimal flexion. Flexible bars were obtained which, when stored at room temperature, showed no oiliness or exudation several weeks after pressing. Two of the sample bars were visually evaluated for appearance and clarity by placing the bars over a standard printed text. The qualitative flexibility of the bars was also crudely evaluated by hand. The bars were placed in aluminum pans which were then placed inside a glass crystallization dish covered with a watch glass. The bars were allowed to sit under ambient conditions at room temperature for over three weeks and re-evaluated during and at the end of this aging period. Table 6 presents appearance values and notes.

TABLE 6
Room Temperature Aging Clarity and Appearance
Properties of Plasticized PVC Bars.

| Material Used | Initial Clarity | Final Clarity | Notes on Bar at |
|---|---|---|---|
| in Bar | Value (Day 9)* | Value (Day 27) | End of Test |
| DINP | 1 | 1 | OK flex |
| C₁₀BzC₅ | 1 | 1 | Good flex, >DINP |

*1-5 scale, 1 = no distortion, 5 = completely opaque.

### Example 6

### Weight Loss Study of Plasticized PVC Bars

Two each of the PVC sample bars prepared in Evaluation Example 2 (C₁₀BzC₅ or DINP) were placed separately in aluminum weighing pans and placed inside a convection oven at 98° C. Initial weight measurements of the hot bars and measurements taken at specified time intervals were recorded and results were averaged between the bars. Additional fresh bars, or alternately a small piece of thin material taken from the mold overflow, were subjected to Thermogravimetric Analysis as described in Example 4 to evaluate plasticizer volatility in the formulated test bars. Results are shown in Table 7. Notes on the appearance and flexibility of the bars at the end of the 98° C. test are also given.

TABLE 7
% Weight Loss by TGA or in 98° C. Oven of Plasticized PVC Bars.

| Material | Oven | Oven | Oven | | TGA | TGA | | % |
|---|---|---|---|---|---|---|---|---|
| Used | Day | Day | Day | | 1% Loss | 5% Loss | TGA 10% | Loss, |
| in Bar | 1 (%) | 7 (%) | 21 (%) | Notes | (° C.) | (° C.) | Loss (° C.) | 220° C. |
| DINP^{a} | 0.20 | 0.30 | 0.62 | Stiff, sl. | 204.6 | 247.4 | 257.6 | 1.8 |
| | | | | curled | | | | |
| C₁₀BzC₅^{b} | 0.12 | 0.58 | 0.65 | Excellent | 189.0 | 231.2 | 246.1 | 3.3 |
| | | | | flex | (185.1) | (225.6) | (245.4) | (4.1) |

^{a}TGA data is from a bar.
^{b}Main TGA data is from a bar, aged 8 days; parenthetical data is from mold overflow film, aged 149 days.

### Example 7

### Humid Aging Study of Plasticized PVC Bars

Using a standard one-hole office paper hole punch, holes were punched in two of the sample bars prepared in Example 4 (DINP or C₁₀BzC₅) ⅛″ from one end of the bar. The bars were hung in a glass pint jar (two bars per jar) fitted with a copper insert providing a stand and hook. The jar was filled with ˜½″ of distilled water and the copper insert was adjusted so that the bottom of each bar was ˜1″ above the water level. The jar was sealed, placed in a 70° C. convection oven, and further sealed by winding Teflon® tape around the edge of the lid. After 21 days the jars were removed from the oven, allowed to cool for ˜20 minutes, opened, and the removed bars were allowed to sit under ambient conditions in aluminum pans (with the bars propped at an angle to allow air flow on both faces) or hanging from the copper inserts for ca. one week (until reversible humidity-induced opacity had disappeared). The bars were evaluated visually for clarity. The bars exhibited complete opacity during the duration of the test and for several days after removal from the oven. Results are summarized in Table 8.

TABLE 8
70° C. Humid Aging Clarity and Appearance
Properties of Plasticized PVC Bars.

| Material Used | Clarity Value After Test* | |
|---|---|---|
| in Bar | (days aged at ambient) | Notes on Bar |
| DINP | 1 | OK flex |
| C₁₀BzC₅ | 1.5 | OK flex/somewhat stiff |

*1-5 scale, 1 = no distortion, 5 = completely opaque. Both sets of bars showed very minor oiliness and white spots/haze (indicating incomplete dehumidification) 30 days after end of test.

### Example 8

### Demonstration of PVC Plasticization by Differential Scanning Calorimetry (DSC) and Dynamic Thermal Mechanical Analysis (DMTA)

Three-point bend Dynamic Mechanical Thermal Analysis (DMTA) with a TA Instruments DMA Q980 fitted with a liquid N₂ cooling accessory and a three-point bend clamp assembly was used to measure the thermo-mechanical performance of neat PVC and the PVC/plasticizer blend sample bars prepared in Example 4. Samples were loaded at room temperature and cooled to −60° C. at a cooling rate of 3° C./minute. After equilibration, a dynamic experiment was performed at one frequency using the following conditions: 3° C./min heating rate, 1 Hz frequency, 20 micrometer amplitude, 0.01 pre-load force, force track 120%. Two or three bars of each sample were typically analyzed; numerical data was taken from the bar typically exhibiting the highest room temperature storage modulus (the bar assumed to have the fewest defects) unless another run was preferred for data quality. Glass transition onset values were obtained by extrapolation of the tan delta curve from the first deviation from linearity. The DMTA measurement gives storage modulus (elastic response modulus) and loss modulus (viscous response modulus); the ratio of loss to storage moduli at a given temperature is tan delta. The beginning (onset) of the T_{g} (temperature of brittle-ductile transition) was obtained for each sample by extrapolating a tangent from the steep inflection of the tan delta curve and the first deviation of linearity from the baseline prior to the beginning of the peak. Table 9 provides a number of DMTA parameters for the bars (the temperature at which the storage modulus equals 100 MPa was chosen to provide an arbitrary measure of the temperature at which the PVC loses a set amount of rigidity; too much loss of rigidity may lead to processing complications for the PVC material). The flexible use temperature range of the samples was evaluated as the range between the T_{g} onset and the temperature at which the storage modulus was 100 MPa. A lowering and broadening of the glass transition for neat PVC was observed upon addition of the OXO ester plasticizer, indicating plasticization. Plasticization (enhanced flexibility) was also demonstrated by lowering of the PVC room temperature storage modulus. Differential Scanning calorimetry (DSC) was also performed on the compression-molded sample bars (−90° C. to 100-170° C. at 10° C./min). Small portions of the sample bars (˜5-7 mg) or, alternately, pieces of thin film, were cut for analysis, making only vertical cuts perpendicular to the largest surface of the bar to preserve the upper and lower compression molding “skins”; the pieces were then placed in the DSC pans so that the upper and lower “skin” surfaces contacted the bottom and top of the pan. Results are summarized in Table 9; lowering and broadening of the glass transition for neat PVC indicates plasticization by the OXO-ester (for aid in calculating the numerical values of these broad transitions, the DSC curve for each plasticized bar was overlaid with the analogous DMTA curve for guidance the proper temperature regions for the onset, midpoint, and end of T_{g}).

TABLE 9
DMTA and DSC Thermal Parameters for Plasticized PVC Bars

| | | | 25° C. | Temp. of | Flex. | | | | Tₘ Max |
|---|---|---|---|---|---|---|---|---|---|
| Material | Tan Δ T_{g} | Tan Δ | Storage | 100 MPa | Use | DSC T_{g} | DSC T_{g} | DSC T_{g} | (° C.), |
| Used | Onset | Peak | Mod. | Storage | Range | Onset | Midpt | End | ΔH_{f} |
| in Bar | (° C.) | (° C.) | (MPa) | Mod. (° C.) | (° C.)^{a} | (° C.) | (° C.) | (° C.) | (J/g)^{b} |
| DINP | −37.6 | 17.1 | 48.6 | 16.9 | 54.5 | −37.8 | −24.8 | −12.2 | N/A^{d} |
| C₁₀BzC₅^{e} | −42.4 | 2.4 | 23.0 | 5.3 | 47.7 | −54.6 (−50.0) | −36.1 | −17.6 | 56.9, 0.9 |
| | | | | | | | (−33.2) | (−26.5) | (62.1, |
| | | | | | | | | | 1.3) |
| None^{c} | 44.0 | 61.1 | 1433 | 57.1 | 13.1 | 44.5 | 46.4 | 48.9 | N/A |

N/A = Not analyzed.
^{a}Difference between DMTA temperature of 100 MPa storage modulus and onset of T_{g}.
^{b}Some sample bars showed a weak melting point (Tₘ) from the crystalline portion of PVC. Often this weak transition was not specifically analyzed, but data is given here in instances where it was recorded.
^{c}Neat PVC, no plasticizer used.
^{d}Very small.
^{e}Main DSC and DMTA data is from a film and bar, respectively, aged ~13 days; parenthetical data is from a bar aged 165 days.

### Example 9

### Further Demonstration of PVC Plasticization with C₁₀BzC₅

Plasticized PVC samples containing C₁₀BzC₅ or DINP (as a comparative) were mixed at room temperature with moderate stirring, then placed on a roll mill at 340° F. and milled for six minutes. The flexible vinyl sheet was removed and compression molded at 340° F. The samples had the following formulation: 100 phr Oxy Vinyls® 240 PVC resin; 60 phr C₁₀BzC₅ or DINP; 20 phr CaCO₃; 2 phr Naftosafe® PKP314 stabilizer. Comparison of the data for the formulations is given in Table 10.

TABLE 10
Properties of PVC Samples Plasticized
with 60 phr C₁₀BzC₅ or DINP

| | C₁₀BzC₅ | DINP |
|---|---|---|
| Original Mechanical Properties | | |
| Shore A Hardness (15 sec.) | 71.9 | 79.8 |
| 95% Confidence Interval | 0.7 | 0.3 |
| Shore D Hardness (15 sec.) | 17.5 | 22.3 |
| 95% Confidence Interval | 0.3 | 0.3 |
| 100% Modulus Strength, psi | 1098 | 1437 |
| 95% Confidence Interval | 8 | 16 |
| Ultimate Tensile Strength, psi | 2800 | 2941 |
| 95% Confidence Interval | 62 | 62 |
| Ultimate Elongation, % | 356 | 370 |
| 95% Confidence Interval | 14 | 11 |
| Aged Mechanical Properties (7 days at 100° C., | | |
| AC/hour) | | |
| Aged 100% Modulus Strength, psi | 2406 | 2007 |
| 95% Confidence Interval | 28 | 18 |
| Ultimate Tensile Strength, psi | 2793 | 2908 |
| 95% Confidence Interval | 50 | 69 |
| Ultimate Elongation, % | 259 | 320 |
| 95% Confidence Interval | 15 | 15 |
| Weight Loss, Wt % | 14.6 | 7.5 |
| 95% Confidence Interval | 0.32 | 0.28 |
| Retained Properties (7 days at 100° C., AC/hour) | | |
| Retained 100% Modulus Strength, % | 219 | 140 |
| 95% Confidence Interval | 0.7 | 0.4 |
| Retained Tensile Strength, % | 100 | 99 |
| 95% Confidence Interval | 0.4 | 0.3 |
| Retained Elongation, % | 73 | 87 |
| 95% Confidence Interval | 1.3 | 1.1 |
| Carbon Volatility (24 hours at 70° C.) | | |
| Mean (3 Specimens) | 0.43 | 0.19 |
| 95% Confidence Interval | 0.03 | 0.03 |
| Low Temperature | | |
| Clash Berg (T_{f}), ° C. | −29.0 | −22.7 |
| 95% Confidence Interval | 1.7 | 1.3 |

The meanings of terms used herein shall take their ordinary meaning in the art; reference shall be taken, in particular, to Handbook of Petroleum Refining Processes, Third Edition, Robert A. Meyers, Editor, McGraw-Hill (2004). In addition, all patents and patent applications, test procedures (such as ASTM methods), and other documents cited herein are fully incorporated by reference to the extent such disclosure is not inconsistent with this disclosure and for all jurisdictions in which such incorporation is permitted. Also, when numerical lower limits and numerical upper limits are listed herein, ranges from any lower limit to any upper limit are contemplated. Note further that Trade Names used herein are indicated by a ™ symbol or ® symbol, indicating that the names may be protected by certain trademark rights, e.g., they may be registered trademarks in various jurisdictions.

The disclosure has been described above with reference to numerous embodiments and specific examples. Many variations will suggest themselves to those skilled in this art in light of the above detailed description. All such obvious variations are within the full intended scope of the appended claims.

What is claimed is:

1. An ester of the formula: 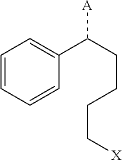
wherein A is either —OC(O)R′ or ═O, and X is either —COC(O)R or —C(O)OR, and R and R′ are C₃ to C₁₃ alkyl, which are the same or different.

2. The ester of claim 1, which is a diester of the formula:

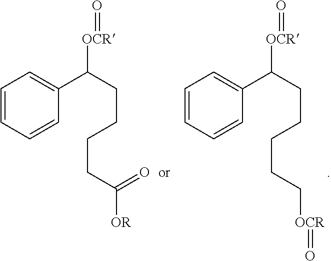

3. A mixture of diesters of the formula: 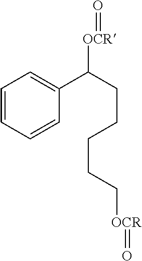
wherein R and R′ are alkyl residues of C₄ to C₁₃ OXO-acids, which are the same or different.

4. The diesters of claim 3, wherein R and R′ are mixed alkyl isomer residues of C₄ to C₁₃ OXO-acids.

5. The diesters of claim 4, wherein R and R′ are mixed alkyl isomer residues of C₄ to C₉ OXO-acids.

6. A mixture of diesters of the formula: 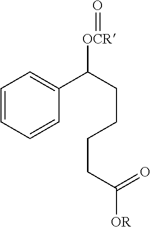
wherein R is alkyl residues of C₄ to C₁₃ OXO-alcohols, and R′ is alkyl residues of C₄ to C₁₃ OXO-acids, and wherein R and R′ can have the same or different carbon numbers.

7. The diesters of claim 6, wherein R and R′ are mixed alkyl isomer residues of the respective OXO-alcohols and OXO-acids.

8. The diesters of claim 6, wherein R is mixed alkyl isomer residues of C₄ to C₉ OXO-alcohols and R′ is mixed alkyl isomer residues of C₄ to C₉ OXO-acids.

9. A composition comprising a polymer and one or more plasticizer of the formula: 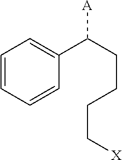
wherein A is either —OC(O)R′ or ═O, and X is either —COC(O)R or —C(O)OR, and R and R′ are C₃ to C₁₃ alkyl, which are the same or different.

10. The composition of claim 9, wherein R and R′ are mixed alkyl isomers.

11. The composition of claim 10, wherein the plasticizer is a mixture of compounds of the formula: 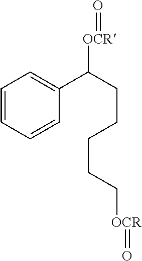
wherein R and R′ are alkyl residues of C₄ to C₁₃ OXO-acids, and wherein R and R′ can have the same or different carbon numbers.

12. The composition of claim 10, wherein the plasticizer is a mixture of compounds of the formula: 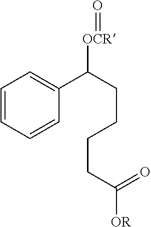
wherein R is alkyl residues of C₄ to C₁₃ OXO-alcohols, and R′ is alkyl residues of C₄ to C₁₃ OXO-acids, and wherein R and R′ can have the same or different carbon numbers.

13. The composition of claim 9, wherein the polymer is selected from the group consisting of vinyl chloride resins, polyesters, polyurethanes, ethylene-vinyl acetate copolymer, rubbers, poly(meth)acrylics and combinations thereof.

14. The composition of claim 13, wherein the polymer is polyvinylchloride.

15. A process for forming 6-hydroxyhexanophenone, comprising:
(a) oxidizing cyclohexylbenzene in the presence of a molecular oxygen containing gas and N-hydroxyphthalimide catalyst to form cyclohexylbenzene hydroperoxide; and
(b) cleaving the cyclohexyl moiety of said cyclohexylbenzene hydroperoxide in the presence of a polar solvent and an acid to form 6-hydroxyhexanophenone.

16. The process of claim 15, wherein said molecular oxygen containing gas is selected from the group consisting of air and oxygen.

17. The process of claim 15, wherein said acid is sulfuric acid.

18. The process of claim 15, wherein said polar solvent is selected from the group consisting of acetone, nitromethane, nitrobenzene, acetonitrile, dimethylsulfoxide, and water.

19. The process of claim 18, wherein said polar solvent is nitromethane.

20. A process for forming 6-hydroxyhexanophenone, comprising:
(a) oxidizing cyclohexylbenzene in the presence of air and N-hydroxyphthalimide catalyst to form cyclohexylbenzene hydroperoxide;
(b) cleaving the cyclohexyl moiety of said cyclohexylbenzene hydroperoxide in the presence of nitromethane and sulfuric acid to form 6-hydroxyhexanophenone.

21. A process of forming diesters of the formula: 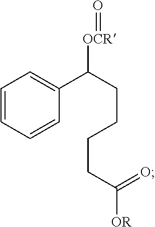
comprising:
(a) oxidizing cyclohexylbenzene in the presence of air and N-hydroxyphthalimide catalyst to form cyclohexylbenzene hydroperoxide;
(b) cleaving the cyclohexyl moiety of said cyclohexylbenzene hydroperoxide in the presence of nitromethane and sulfuric acid to form 6-hydroxyhexanophenone;
(c) oxidizing said 6-hydroxyhexanophenone to form a mono-acid of the formula: 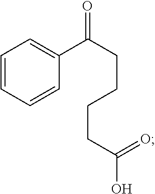
(d) esterifying said mono-acid with alcohols of the formula ROH, wherein R is C₄ to C₁₃ alkyl which can be the same or different to form monoesters of the formula: 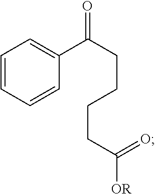
(e) hydrogenating said monoester to form a compound of the formula: 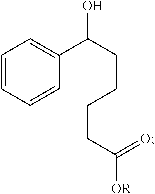
and
(f) esterifying the hydroxyl group with a second carboxylic acid of the formula R′C(O)OH, wherein R′ is C₄ to C₁₃ alkyl, which can be the same or different, to form a diester of the formula: 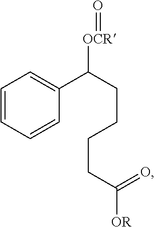
wherein R and R′ are C₃ to C₁₃ alkyl, and can have the same or different carbon numbers.

22. The process of claim 21, wherein R is mixed alkyl isomer residues of C₄ to C₁₃ OXO-alcohols, and R′ is mixed alkyl isomer residues of C₄ to C₁₃ OXO-acids.

23. A process of forming diesters of the formula: 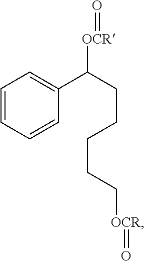
comprising:
(a) oxidizing cyclohexylbenzene in the presence of air and N-hydroxyphthalimide catalyst to form cyclohexylbenzene hydroperoxide;
(b) cleaving the cyclohexyl moiety of said cyclohexylbenzene hydroperoxide in the presence of nitromethane and sulfuric acid to form 6-hydroxyhexanophenone;
(c) esterifying said 6-hydroxyhexanophenone with a first carboxylic acid of the formula ROOH, wherein R is C₄ to C₁₃ alkyl, which can be the same or different, to form monoesters of the formula: 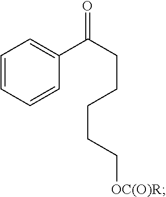
(d) hydrogenating said monoester to form a compound of the formula: 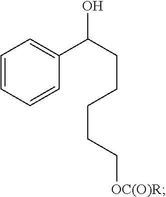
and
(e) esterifying the hydroxyl group with a carboxylic acid of the formula R′C(O)OH, wherein R′ is C₄ to C₁₃ alkyl, which can be the same or different, to form a diester of the formula: 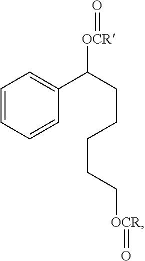
wherein R and R′ are C₃ to C₁₃ alkyl, and can have the same or different carbon numbers.

24. The process of claim 23, wherein R and R′ are alkyl residues of C₄ to C₁₃ OXO-acids.

25. The process of claim 24, wherein R and R′ are mixed alkyl isomer residues of the OXO-acids.

26. A monoester of the formula: 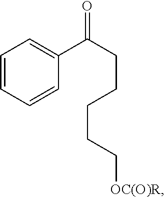
wherein R is C₃ to C₁₃ alkyl which can be the same or different.

27. A mixture of monoesters of the formula: 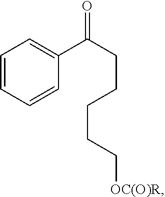
wherein R is C₃ to C₁₃ alkyl which can be the same or different.

28. The monoesters of claim 27, wherein R is mixed alkyl isomer residues of C₄ to C₁₃ OXO-acids.

29. The monoesters of claim 28, wherein R is mixed alkyl isomer residues of C₄ to C₉ OXO-acids.

30. A monoester of the formula: 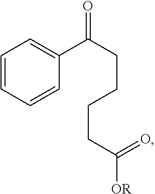
wherein R is C₄ to C₁₃ alkyl which can be the same or different.

31. A mixture of monoesters of the formula: 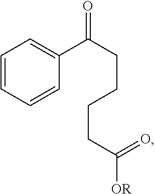
wherein R is C₄ to C₁₃ alkyl which can be the same or different.

32. The monoesters of claim 31, wherein R is mixed alkyl isomer residues of C₄ to C₁₃ OXO-alcohols.

33. The monoesters of claim 32, wherein R is mixed alkyl isomer residues of C₄ to C₉ OXO-alcohols.

34. A composition comprising a polymer and one or more plasticizer of the formula: 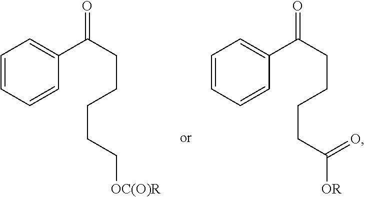
wherein R is C₃ to C₁₃ alkyl, which are the same or different.

35. The composition of claim 34, wherein R is mixed alkyl isomers.

36. The composition of claim 34, wherein R is mixed alkyl isomer residues of C₄ to C₁₃ OXO-acids or OXO-alcohols.

37. The composition of claim 34, wherein the plasticizer is a mixture of compounds of the formula: 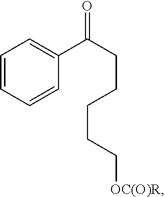
wherein R is mixed alkyl isomer residues of C₄ to C₁₃ OXO-acids.

38. The composition of claim 34, wherein the plasticizer is a mixture of compounds of the formula: 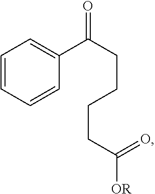
wherein R is mixed alkyl isomer residues of C₄ to C₁₃ OXO-alcohols.

39. The composition of claim 34, wherein the polymer is selected from the group consisting of vinyl chloride resins, polyesters, polyurethanes, ethylene-vinyl acetate copolymer, rubbers, poly(meth)acrylics and combinations thereof.

40. The composition of claim 39, wherein the polymer is polyvinylchloride.
